(19) United States
(12) Patent Application Publication
(10) Pub. No.: US 2025/0295414 A1
(43) Pub. Date: Sep. 25, 2025

(54) Left Atrial Appendage Occluder Hook Placement

(71) Applicant: St. Jude Medical, Cardiology Division, Inc., St. Paul, MN (US)

(72) Inventors: Sadie Cronin, Plymouth, MN (US); Gary Erzberger, Corcoran, MN (US); Andrey Kai, Plymouth, MN (US); Matthew Arthur Tanner, Minneapolis, MN (US); Trevor J. Springer, Maple Grove, MN (US); Sarah Grenell, Watertown, MN (US); Kaycee Peek, Maple Grove, MN (US)

(73) Assignee: St. Jude Medical, Cardiology Division, Inc., St. Paul, MN (US)

(21) Appl. No.: 18/965,997

(22) Filed: Dec. 2, 2024

Related U.S. Application Data
(60) Provisional application No. 63/569,321, filed on Mar. 25, 2024.

(51) Int. Cl. A61B 17/12

(52) U.S. Cl. CPC A61B 17/12122; A61B 17/12145

(57) ABSTRACT

A left atrial appendage (“LAA”) occluder may include a lobe with proximal and distal ends defining proximal and distal surfaces of the lobe, respectively, and a middle portion therebetween, the lobe having an expanded unbiased condition and a collapsed delivery condition. A plurality of stabilizing wires may be coupled to the lobe, each wire having a hook at a distal end thereof, each hook having a terminal end positioned radially outwardly of the lobe in the expanded condition of the lobe and being configured to frictionally engage tissue of the LAA. In the expanded condition of the lobe, each hook has a distalmost point being positioned a spaced axial distance from the distal surface of the lobe in the expanded condition of the lobe, the spaced axial distance being between about 0 mm and about 1.5 mm.

### CROSS-REFERENCE TO RELATED APPLICATIONS

This application claims priority to the filing date of U.S. Provisional Patent Application No. 63/569,321, filed Mar. 25, 2024, the disclosure of which is hereby incorporated by reference herein.

### BACKGROUND OF THE DISCLOSURE

An occluder is a medical device used to treat (e.g., occlude) tissue at a target site within the human body, such as an abnormality, a vessel, an organ, an opening, a chamber, a channel, a hole, a cavity, a lumen, or the like. For example, an occluder may be used for left atrial appendage (“LAA”) closures. An LAA is a normal anatomical structure in which there is a sac in the muscle wall of the left atrium. When a patient experiences atrial fibrillation (“AFib”), a blood clot may be formed within the LAA which may become dislodged and enter into the blood stream. By occluding the LAA, the release of blood clots from the LAA may be significantly reduced, if not eliminated. Various techniques have been developed to occlude the LAA. For instance, balloon-like devices have been developed that are configured to be implanted completely within the cavity of the LAA, while surgical techniques have also been developed where the cavity of the LAA is inverted and surgically closed.

### SUMMARY OF THE DISCLOSURE

According to one aspect of the disclosure, an occluder for occluding a left atrial appendage (“LAA”) may include a lobe sized and shaped for positioning within the LAA, the lobe having a proximal end defining a proximal surface of the lobe, a distal end defining a distal surface of the lobe, and a middle portion connecting and extending between the proximal end and the distal end of the lobe, wherein the lobe has an expanded condition in the absence of applied forces and a collapsed condition for delivery to the LAA. The occluder may include a plurality of stabilizing wires coupled to the lobe, each of the plurality of stabilizing wires having a hook at a distal end thereof, each hook having a terminal end positioned radially outwardly of the lobe in the expanded condition of the lobe and being configured to frictionally engage tissue of the LAA. In the expanded condition of the lobe, each hook may have a distalmost point, the distalmost point of each hook being positioned a spaced axial distance from the distal surface of the lobe in the expanded condition of the lobe, the spaced axial distance being between about 0 mm and about 1.5 mm. The spaced axial distance may be about 1.0 mm. A transition between the middle portion and the distal end of the lobe may be curved, and the curve of the transition may have a contour length measured between (i) a point at which a distal end of the middle portion joins a proximal end of the curved transition, and (ii) a point at which a distal end of the curved transition joins the distal surface of the lobe. In the expanded condition of the lobe, the hook may cross from an interior of the lobe to an exterior of the lobe at a crossing point, the crossing point being positioned between about 25% and about 50% of the contour length. The transition may have a radius of curvature between about 0.075 inches (1.905 mm) and about 0.125 inches (3.175 mm). The occluder may include a disc at a proximal end of the occluder, and a connecting member may connect the disc and the lobe. In a compressed state of the lobe, a valley may be formed within the middle portion of the lobe, and in the compressed state, the hook may extend farther radially outward from a central longitudinal axis of the lobe than does any structure of the lobe.

According to another aspect of the disclosure an occluder for occluding a left atrial appendage (“LAA”), may include a lobe sized and shaped for positioning within the LAA, the lobe having a proximal end defining a proximal surface of the lobe, a distal end defining a distal surface of the lobe, and a middle portion connecting and extending between the proximal end and the distal end of the lobe, wherein the lobe has an expanded condition in the absence of applied forces and a collapsed condition for delivery to the LAA. A plurality of stabilizing wires may be coupled to the lobe, each of the plurality of stabilizing wires having a hook at a distal end thereof, each hook having a terminal end positioned radially outwardly of the lobe in the expanded condition of the lobe and being configured to frictionally engage tissue of the LAA. A transition between the middle portion and the distal end of the lobe may be curved, the curve of the transition having a contour length measured between (i) a point at which a distal end of the middle portion joins a proximal end of the curved transition, and (ii) a point at which a distal end of the curved transition joins the distal surface of the lobe. In the expanded condition of the lobe, the hook may cross from an interior of the lobe to an exterior of the lobe at a crossing point, the crossing point being positioned between about 25% and about 50% of the contour length. In the expanded condition of the lobe, the crossing point may be positioned between about 25% and about 40% of the contour length. In the expanded condition of the lobe, the crossing point may be positioned between about 30% and about 35% of the contour length. In the expanded condition of the lobe, each hook may have a distalmost point, the distalmost point of each hook being positioned a spaced axial distance from the distal surface of the lobe in the expanded condition of the lobe, the spaced axial distance being about 1 mm. The transition may have a radius of curvature between about 0.075 inches (1.905 mm) and about 0.125 inches (3.175 mm). The occluder may include a disc at a proximal end of the occluder, and a connecting member may connect the disc and the lobe. In a compressed state of the lobe, a valley may be formed within the middle portion of the lobe, and in the compressed state, the hook may extend farther radially outward from a central longitudinal axis of the lobe than does any structure of the lobe.

According to a further aspect of the disclosure, a method for occluding a left atrial appendage (“LAA”) may include positioning an occluder in a collapsed condition within a delivery catheter. The occluder may include a lobe having a proximal end defining a proximal surface of the lobe, a distal end defining a distal surface of the lobe, and a middle portion connecting and extending between the proximal end and the distal end of the lobe. The occluder may also include a plurality of stabilizing wires coupled to the lobe, each of the plurality of stabilizing wires having a hook at a distal end thereof. The method may further include advancing the occluder into or adjacent to the LAA while the occluder is collapsed within the delivery catheter. The method may further include transitioning the lobe into a partially-expanded state in which the distal end of the lobe has exited the delivery catheter and has begun to self-expand while the proximal end of the lobe remains collapsed within the delivery catheter. While the lobe is in the partially expanded state, the hook may be contacted with tissue of the LAA. After contacting the hook with tissue of the LAA, the occluder may be fully deployed from the delivery catheter. Between the partially expanded state of the lobe and the full deployment of the occluder from the delivery catheter, the hook may remain in contact with the tissue of the LAA. After fully deploying the occluder from the delivery catheter, compression on the lobe from tissue of the LAA may create a valley within the middle portion of the lobe. While the valley is created within the middle portion of the lobe, the hook may remain in contact with the tissue of the LAA. Fully deploying the occluder from the delivery catheter may include deploying a disc of the occluder from the delivery catheter, the disc of the occluder being coupled to the lobe of the occluder by a connecting member. Upon deploying the disc of the occluder, the disc may at least partially cover an ostium of the LAA.

### BRIEF DESCRIPTION OF THE DRAWINGS

FIG. 1 illustrates an occluder according to the prior art.

FIG. 2 illustrates a lobe of the occluder of FIG. 1 in a deployed, over-compressed state, with a disc of the occluder being out of the view of FIG. 2.

FIG. 3 illustrates the occluder of FIG. 1 mid-deployment.

FIG. 4 is a schematic diagram of a delivery system in accordance with the present disclosure.

FIGS. 5 and 6 are illustrations and pictures, respectively, of a side view of an exemplary embodiment of a medical device including a rounded lobe, in accordance with the present disclosure.

FIG. 7 illustrates the lobe of the occluder of FIGS. 5-6 in a deployed, over-compressed state, with a disc of the occluder being out of the view of FIG. 7.

FIG. 8 illustrates the occluder of FIGS. 5-6 mid-deployment.

FIG. 9 is an enlarged view of a portion of the lobe of the occluder of FIGS. 5-6.

FIG. 10 is an isolated view of an example of a stabilizing wire of the occluder of FIGS. 5-6.

FIG. 11 is a flow diagram of an exemplary method of using the medical device of FIGS. 5-6 to occlude an LAA in a patient.

FIG. 12 is a flow diagram of an exemplary method of fabricating the medical device of FIGS. 5-6.

### DETAILED DESCRIPTION OF THE DISCLOSURE

LAA occlusion procedures that employ a transcatheter occluder typically require accurate deployment of the occluder into a highly variable anatomical feature. LAA structures can be highly variable from patient to patient. For example, LAA structures can vary in diameter, length, orientation, number of lobes, ovality, bend angle, etc. At least in part because of this variability, determining the appropriate size of a self-expandable transcatheter occluder device to occlude the LAA may be particularly difficult.

Although it may be possible to estimate the proper size of the occluder for use in a particular patient's LAA, true assessment of the occluder's fit in the LAA cannot be determined until the occluder is actually implanted into the LAA. If the incorrect occluder size is selected, the occluder may not fit into the LAA or the occluder may not anchor properly within the LAA. This can lead to embolization of the occluder, or the need to retrieve the occluder and implant another occluder having a different size. Thus, it would be desirable to have an occluder device that works well in a wider range of anatomies, which may lessen the criticality of selecting the correct size occluder for the particular patient.

Additionally, when deploying a self-expandable transcatheter occluder from a delivery device, stability of the occluder during deployment can be an important factor in being able to accurately place the occluder within the LAA. Thus, it would be desirable to have an occluder device that is able to engage with LAA tissue relatively early in deployment to help stabilize the occluder in the desired position within the LAA before fully deploying the occluder.

The present disclosure relates generally to medical devices that are used in the human body. In particular, the present disclosure is directed to an occlusion device having hook placement and frame profile that allow for enhanced stability of the occluder during deployment as well as the ability for a particular size of the occluder to work in a wide range of anatomies.

The disclosed embodiments may lead to more consistent and improved patient outcomes. It is contemplated, however, that the described features and methods of the present disclosure as described herein may be incorporated into any number of systems as would be appreciated by one of ordinary skill in the art based on the disclosure herein.

Although the exemplary embodiment of the medical device is described as treating a target site including a LAA, it should be understood that the use of the term “target site” is not meant to be limiting, as the medical device may be configured to treat any target site, such as an abnormality, a vessel, an organ, an opening, a chamber, a channel, a hole, a cavity, or the like, located anywhere in the body. The term “vascular abnormality,” as used herein is not meant to be limiting, as the medical device may be configured to bridge or otherwise support a variety of vascular abnormalities. For example, the vascular abnormality could be any abnormality that affects the shape of the native lumen, such as an atrial septal defect, a lesion, a vessel dissection, or a tumor. Embodiments of the medical device may be useful, for example, for occluding a patent foramen ovalis (“PFO”), atrial septal defect (“ASD”), ventricular septal defect (“VSD”), or patent ductus arteriosus (“PDA”), as noted above. Furthermore, the term “lumen” is also not meant to be limiting, as the vascular abnormality may reside in a variety of locations within the vasculature, such as a vessel, an artery, a vein, a passageway, an organ, a cavity, or the like. As used herein, the term “proximal” refers to a part of the medical device or the delivery device that is closest to the operator when the device is being used as intended, and the term “distal” refers to a part of the medical device or the delivery device that is farther from the operator at any given time as the medical device is being delivered through the delivery device. In addition, the terms “deployed” and “implanted” may be used interchangeably herein.

Some embodiments of the present disclosure provide an improved percutaneous catheter directed intravascular occlusion device for use in the vasculature in patients' bodies, such as blood vessels, channels, lumens, a hole through tissue, cavities, and the like, such as an ASD. Other physiologic conditions in the body occur where it is also desirous to occlude a vessel or other passageway to prevent blood flow into or therethrough. These device embodiments may be used anywhere in the vasculature where the anatomical conditions are appropriate for the design.

The medical device may include one or more layers of occlusive material, wherein each layer may be comprised of any material that is configured to substantially preclude or occlude the flow of blood so as to facilitate thrombosis. As used herein, “substantially preclude or occlude flow” shall mean, functionally, that blood flow may occur for a short time, but that the body's clotting mechanism or protein or other body deposits on the occlusive material results in occlusion or flow stoppage after this initial time period.

Some embodiments of the present disclosure may be formed by a plurality of wire strands having a predetermined relative orientation with respect to one another. However, it is understood that according to additional embodiments of the present disclosure, that the medical device could be etched or laser cut from a tube, or the device could comprise an occlusion material coupled to a scaffolding structure or a plurality of slices of a tubular member coupled together.

The present disclosure now will be described more fully hereinafter with reference to the accompanying drawings, in which some, but not all embodiments of the disclosure are shown. Indeed, this disclosure may be embodied in many different forms and should not be construed as limited to the embodiments set forth herein; rather, these embodiments are provided so that this disclosure will satisfy applicable legal requirements. Like numbers refer to like elements throughout.

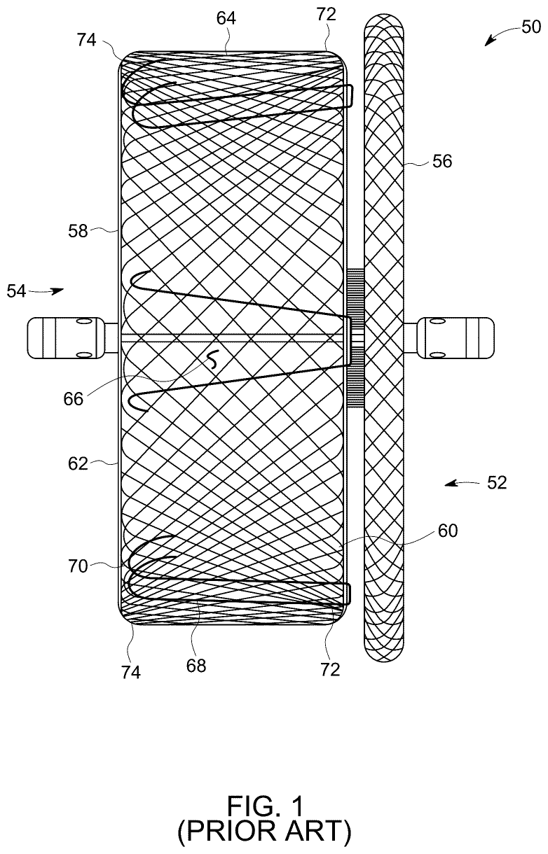

In at least one known medical device used for the occlusion of abnormalities, such as a medical device 50 shown in FIG. 1, medical device 50 includes a proximal end 52 and a distal end 54, with a disc 56 at proximal end 52 and a lobe 58 at distal end 54. The lobe 58 has a proximal edge 60 (also referred to as a proximal face), a distal edge 62 (also referred to as a distal face), and a middle or central portion 64 (“middle portion” and “central portion” may be used interchangeably herein) that define a cavity 66. The medical device 50 also includes stabilizing wires 68 (which may alternatively be referred to as anchors herein) secured to a radially outer or circumferential surface of middle portion 64. The stabilizing wires 68 terminate in a hook 70 at free ends thereof, and thereby facilitate retention of the medical device 50 at a target site and preventing the medical device 50 from becoming dislodged from the target site after deployment.

In this known medical device 50, proximal edge 60 and distal edge 62 adjoin middle portion 64 at a first relatively sharp (i.e., non-rounded) transition 72 and a second sharp transition 74, respectively. First transition 72 connects proximal edge 60 to middle portion 64 by an approximately 90 degree angle. Likewise, second transition 74 connects distal edge 62 to middle portion 64 by an approximately 90 degree angle. First transition 72 and second transition 74 partially define a generally rectangular cross section of lobe 58, leading to relatively sharp circumferential edges of the device and relatively high radial force applied to the surrounding tissue. Stated in another way, first transition 72 and second transition 74 may each have a small radius of curvature, for example between about 0 mm and about 2 mm, including between about 0.5 mm and about 1 mm, including between about 0.6 mm and about 0.8 mm. In the illustrated example, the hooks 70 are positioned proximal to the bend at second transition 74.

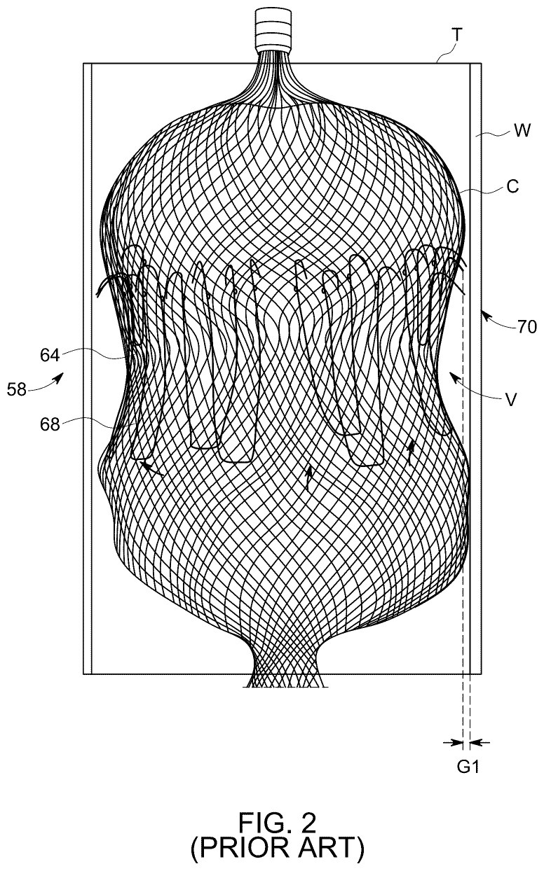

One potential concern with medical device 50 is illustrated in FIG. 2. FIG. 2 shows a 25 mm version of medical device 50 in a state of over-compression, with the lobe 58 being visible but the disc 56 being out of view. In this example, 25 mm refers to the diameter of the lobe 58 in the unbiased state or in the absence of applied forces. As used herein, the term over-compression may generally refer to a situation in which the medical device 50 is deployed into a cavity or anatomy that is smaller than the indicated use range for the particular medical device 50. For example, the 25 mm version of medical device 50 shown in FIG. 2 may be intended for use in a left atrial appendage having a landing zone width of between 19 mm and 22 mm. In this example, FIG. 2 shows the 25 mm version of medical device 50 being deployed in a cavity that is smaller than 19 mm, for example about 17 mm. In FIG. 2, the lobe 58 of the medical device 50 has been positioned within a cylindrical tube T having a wall W. The outer contour C of the lobe 58, labeled on the right side of FIG. 2, illustrates the contour C of the outer surface of the lobe 58 relative to the wall W of the tube T. As can be seen in FIG. 2, in this state of over-compression, a valley V is created at the middle or central portion 64 of the lobe 58, the valley V being positioned a distance radially inward from the wall W of the tube T, with portions of the lobe 58 proximal and distal to the valley V contacting the inner surface of the wall W of the tube T. For example, in the view of FIG. 2, the portion of the lobe 58 proximal to the valley V on the right side (e.g., the bottom right of the figure) is in contact with the inner wall W of the tube T, but none of the hooks 70 are in contact with the inner wall W of the tube T. It should be understood that the tube T is representative of implantation or deployment in a body cavity, such as the LAA. Notably, the valley V is formed where the hooks 70 of the stabilizing wires 68 exit the lobe 58. In the illustrated state of over-compression, the portion of the medical device 50 at the radially-outer-most position is not the hooks 70, but rather portions of the lobe 58 proximal and/or distal to the valley V. Thus, in this state of over-compression, while the lobe 58 may contact the inner wall W of the tube T (which represents contact with the inner surface of tissue defining a cavity such as the LAA), one or more of the hooks 70 of the stabilizing wires 68 are still spaced away from, and thus not in contact with, the inner wall W of the tube T (which represents lack of contact between the hooks 70 and the tissue of the cavity being treated). This gap is represented in FIG. 2 as gap G1. In the condition shown in FIG. 2, the hooks 70 of the stabilizing wires 68 are unable to engage (or unable to completely engage) with tissue. This phenomenon described above is the result, at least in part, of the location of the stabilizing wires 68 on the lobe 58 as well as the shape of the lobe 58 being generally cylindrical with small-radiused bends at the first transition 72 and the second transition 74. As should be clear from the above description, this phenomenon may result in sub-optimal anchoring of the medical device 50 within the LAA (or within other anatomy being treated by the medical device 50), which may increase the likelihood of the medical device 50 dislodging.

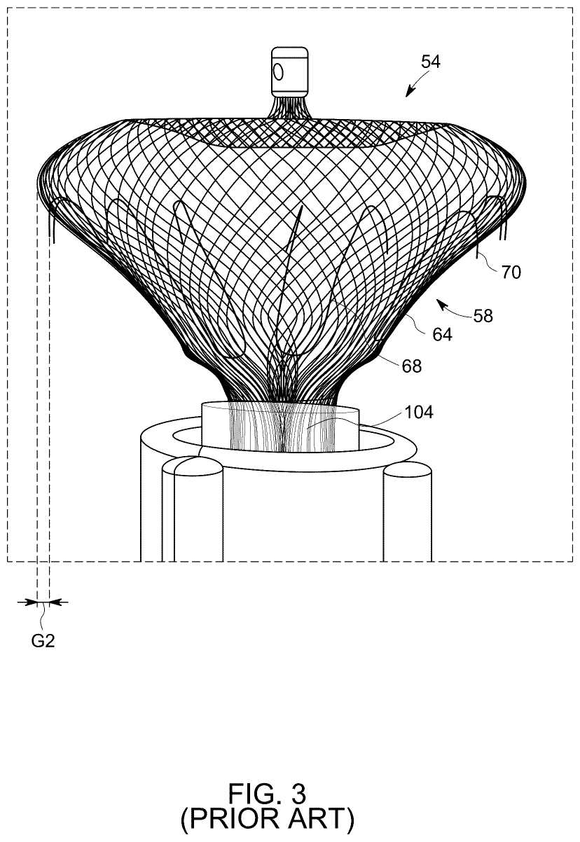

Another potential concern with medical device 50, having some relation to that shown and described in connection with FIG. 2, is illustrated in FIG. 3. FIG. 3 shows the same 25 mm version of medical device 50 in a mid-stage of deployment from a catheter 104. As the leading or distal end 54 of the lobe 58 is expressed from the catheter 104, it begins to self-expand to its set-shape. As can be seen in FIG. 3, in this stage of mid-deployment, while the proximal end 52 of the lobe 58 is maintained within a collapsed condition within catheter 104, the distal end 54 of the medical device 50 extends radially outward from the central longitudinal axis of the medical device 50 farther than any hook 70 of any stabilizing wire 68. This results in another gap G2 between the outer-most point of the frame of the medical device 50 compared to the outer-most point of a hook 70 of the medical device 50. Similar to gap G1, gap G2 may prevent one or more of the hooks 70 from engaging tissue of the cavity (e.g., the LAA wall) during this mid-stage of deployment. This may result in some or none of the hooks 70 being able to engage tissue during deployment until the proximal end 52 of the lobe 58 is fully released (e.g., fully deployed) from the catheter 104. If, when the lobe 58 is being deployed into a cavity, one or more or all hooks 70 are unable to anchor into tissue, the medical device 50 may “jump” (e.g., move and/or reposition rapidly due at least in part to forces of self-expansion) as the lobe 58 is fully deployed due to lack of anchoring, which is undesirable. The concern described in connection to FIG. 3 may apply for all deliveries and deployments of medical device 50, whereas the concern described in connection to FIG. 2 may be more isolated to conditions in which the medical device 50 is over-compressed upon full deployment.

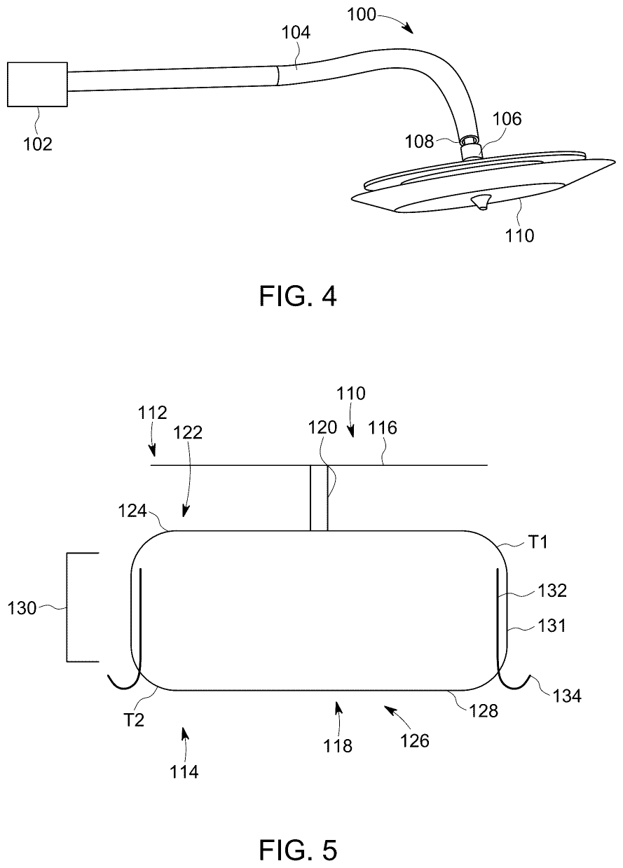

FIG. 4 is a schematic diagram of a delivery system 100. Delivery system 100 may include a delivery device 102 including a catheter 104 and a coupling member 106 configured to couple a distal end of a delivery cable 108 to a medical device 110 for facilitating the deployment of medical device 110 at a target site. Medical device 110 is deployed to treat the target site, and, in the example embodiment, is an occlusion device (“occluder”). It should be understood that delivery system 100 may also be suitable for use with other medical devices, for example including medical device 50.

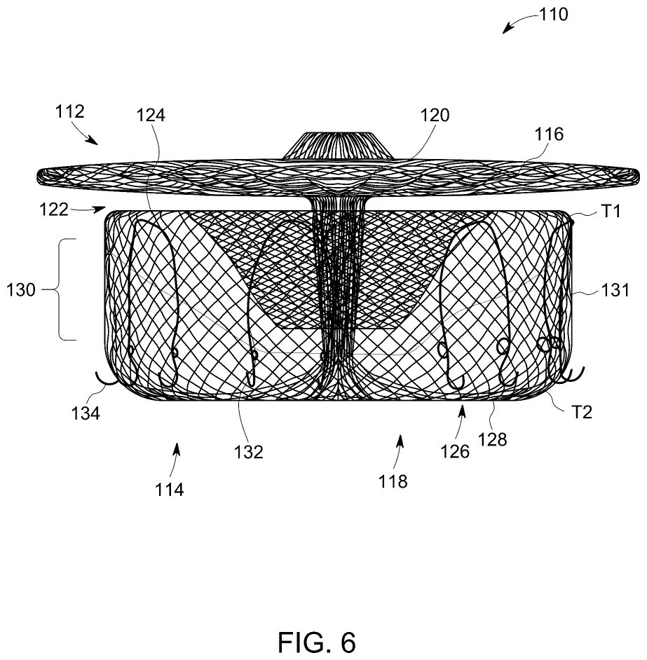

FIGS. 5-6 show an exemplary embodiment of medical device 110. Medical device 110 includes a proximal end 112 and a distal end 114. Proximal end 112 includes a disc 116, and distal end 114 includes a lobe 118, wherein disc 116 and lobe 118 are connected by a connecting member 120. Lobe 118 includes a proximal portion 122 defining a proximal surface 124 of lobe 118, a distal portion 126 defining a distal surface 128 of lobe 118, and a middle or central portion 130 connecting and extending between proximal portion 122 and distal portion 126. Central portion 130 has a circumferential or radially outer surface 131. Proximal surface 124 is connected to or adjoins middle portion 130 at a first transition T1, and distal surface 128 is connected to or adjoins middle portion 130 at a second transition T2. First transition T1 and second transition T2 may each independently have a radius of curvature of between about 0.001 inches (0.0254 mm) and about 0.150 inches (3.81 mm), more particularly between about 0.025 inches (0.635 mm) and about 0.150 inches (3.81 mm), even more particularly between about 0.075 inches (1.905 mm) and about 0.125 inches (3.175 mm). The radius of curvature of first transition T1 may be the same as the radius of curvature of second transition T2. Alternatively, first transition T1 and second transition T2 may have different radii of curvature. In one alternative embodiment, only one of first transition T1 and second transition T2 has a defined radius of curvature, and the other is a relatively sharp transition (e.g., approximately) 90°. With this configuration, the lobe 118 may be generally cylindrical, with radiused bends at the proximal and distal end thereof, although the bends may have different radii of curvature as noted above.

The lobe 118 may further include a plurality of stabilizing wires 132 coupled to lobe 118 at radially outer surface 131 (also referred to as circumferential surface) of middle portion 130. Stabilizing wires 132 may each include a hook 134 at a terminal end thereof. Hooks 134 may extend radially outward from middle portion 130 of lobe 118.

Some embodiments of medical device 110 of the present disclosure may be formed from a braided fabric or mesh material including a plurality of wire strands having a predetermined relative orientation with respect to one another. However, it should be understood that according to additional embodiments of the present disclosure, medical device 110 could be etched or laser cut from a tube, or the device could comprise an occlusion material coupled to a scaffolding structure or frame.

In one embodiment, medical device 110 is formed from a shape-memory material including a metal fabric. The metal fabric is deformed to generally conform to a surface of a mandrel. While on the surface of the mandrel, the metal fabric is treated under heated conditions to allow for the heat-setting of the metal fabric. The heat-setting of the metal fabric ensures that the metal fabric will retain the substantial shape of the mandrel once it is removed from the surface of the mandrel. In the exemplary embodiment, the mandrel utilized for the heat-setting treatment defines the radii of curvature adopted by the metal fabric for the edges of lobe 118 of medical device 110, specifically first transition T1 and second transition T2.

The radius of curvature selected and defined for each of first transition T1 and second transition T2 rounds or softens the circumferential edges of medical device 110. This rounding or softening of the circumferential edge leads to a reduction in the radial force applied to the surrounding tissue. Therefore, medical device 110 is more conformable to the anatomy of the target site in which it is deployed, specifically an LAA.

Disc 116 of medical device 110 is configured to abut the adjacent wall surrounding the opening of the vascular defect to prevent movement of medical device 110 and to assist in sealing of the abnormality in which medical device 110 is deployed. Different sizes and shapes of the disc are contemplated. In one embodiment, the disc portion may be larger in diameter than the vascular abnormality to be occluded to be capable of overlying the opening of the abnormality.

Lobe 118 of medical device 110 is formed to have a suitable size to engage with the lumen of the abnormality that is to be occluded. Medical device 110 may then be held at the target site by radial engagement between lobe 118 and the lumen of the abnormality. Hooks 134 of stabilizing wires 132 also engage with the surrounding tissue and improve retention of medical device 110 at the target site.

One particular shape memory material that may be used to form medical device 110 (and, particularly, lobe 118) as described herein is Nitinol. Nitinol alloys are highly elastic and are said to be “superelastic,” or “pseudoelastic.” This elasticity may allow medical device 110 to be resilient and return to a preset, expanded configuration for deployment following passage in a distorted form through delivery catheter 104. Further examples of materials and manufacturing methods for medical devices with shape memory properties are provided in U.S. Publication No. 2007/0265656 titled “Multi-layer Braided Structures for Occluding Vascular Defects” and filed on Jun. 21, 2007, which is incorporated by reference herein in its entirety.

It is also understood that medical device 110 may be formed from various materials other than Nitinol that have elastic properties, such as stainless steel, trade named alloys such as Elgiloy®, or Hastalloy, Phynox®, MP35N, CoCrMo alloys, metal, polymers, or a mixture of metal(s) and polymer(s). Suitable polymers may include PET (Dacron), polyester, polypropylene, polyethylene, HDPE, Pebax, nylon, polyurethane, silicone, PTFE, polyolefins and ePTFE. Additionally, it is contemplated that the medical device may comprise any material that has the desired elastic properties to ensure that the device may be deployed, function as an occluder as disclosed within this application.

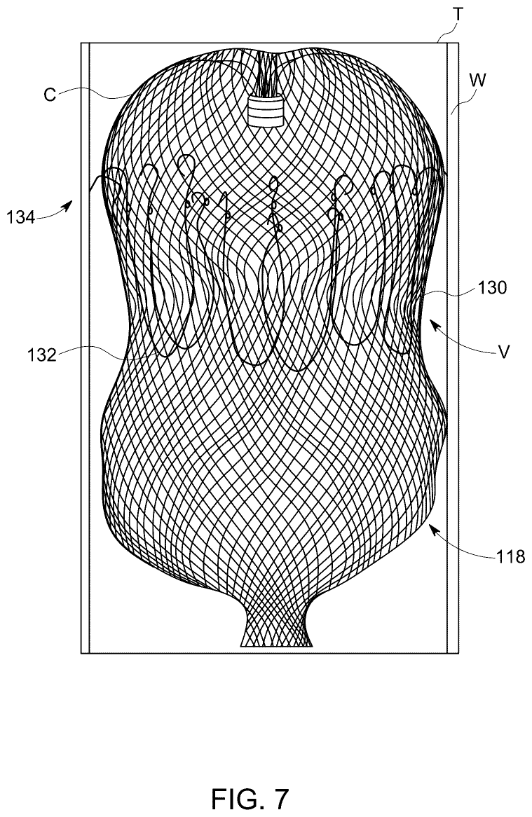

FIG. 7 shows a 25 mm version of medical device 110 in a state of over-compression. In FIG. 7, the lobe 118 of the medical device 110 has been positioned within the same cylindrical tube T as shown and described in connection with FIG. 2. The outer contour C of the lobe 118 is labelled on the left side of FIG. 7 to better illustrate the contour C of the outer surface of the lobe 118 relative to the wall W of the tube T. As can be seen in FIG. 7, in this state of over-compression, a valley V is created at the middle or central portion 130 of the lobe 118, the valley V being positioned a distance radially inward from the wall W of the tube T, with portions of the lobe 118 proximal and distal to the valley V contacting the inner surface of the wall W of the tube T. As described in connection with FIG. 2, it should be understood that the tube T is representative of implantation or deployment in a body cavity, such as the LAA.

Notably, unlike the configuration shown and described in connection with FIG. 2, the valley V is formed a distance proximal to where the hooks 134 of the stabilizing wires 132 exit the lobe 118. Thus, in the illustrated state of over-compression, the portion of the medical device 110 at the radially-outer-most position is the hooks 134, rather than the portions of the lobe 118 proximal and/or distal to the valley V. In this state of over-compression, while the lobe 118 may contact the inner wall W of the tube T (which represents contact with the inner surface of tissue defining a cavity such as the LAA), one or more of the hooks 134 of the stabilizing wires 132 are also in contact with the inner wall W of the tube T (which represents contact between the hooks 134 and the tissue of the cavity being treated). For example, in the view of FIG. 7, although the portions of the lobe 118 proximal and distal to the valley V on the right side are in contact with the inner wall W of the tube T, the hooks 134 on the right side are also in contact with the inner wall W of the tube T. On the other hand, while neither of the portions of the lobe 118 proximal and distal to the valley V on the left side are in contact with the inner wall W of the tube T, the hooks 134 on the left side are in contact with that inner wall W of the tube T. Thus, whereas the configuration shown and described in connection with FIG. 2 includes a gap G1, no such gap exists in the configuration of FIG. 7. In other words, in the condition shown in FIG. 7, the hooks 134 of the stabilizing wires 132 are still able to engage with tissue, despite the lobe 118 being in a state of over-compression. As should be clear from the above description, this phenomenon may result in suitable anchoring of the medical device 110 within the LAA (or within other anatomy being treated by the medical device 110), even in a state of over-compression, which may decrease the likelihood of the medical device 110 dislodging and increase the range of target site (e.g., LAA) sizes that can be appropriately treated with a single size of the medical device 110.

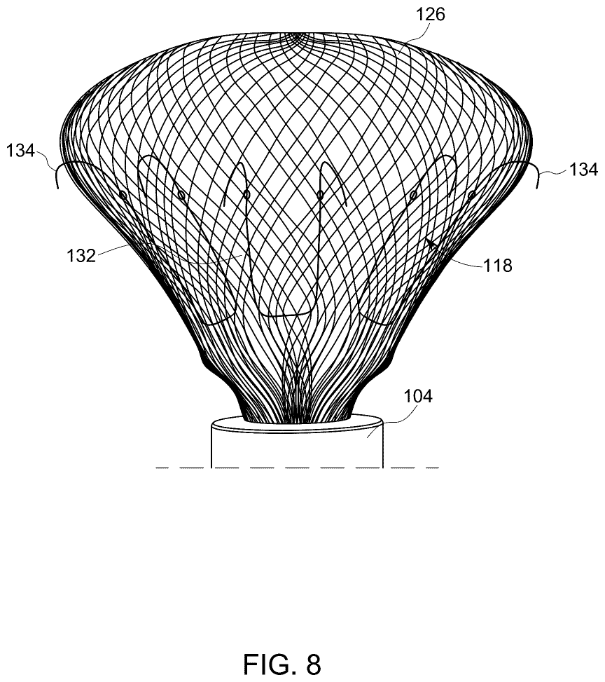

FIG. 8 shows the same 25 mm version of medical device 110 as FIG. 7, but in a mid-stage of deployment from catheter 104. As the leading or distal end 126 of the lobe 118 is expressed from the catheter 104, it begins to self-expand to its set-shape. As can be seen in FIG. 8, in this stage of mid-deployment, while the proximal end 122 of the lobe 118 is maintained within a collapsed condition within catheter 104, the distal end 126 of the medical device 110 extends radially outward from the central longitudinal axis of the medical device 110 farther than most portions of the medical device 110, with the exception of the hooks 134 of the stabilizing wires 132. In other words, compared to the mid-deployment condition of medical device 50 shown in FIG. 3 in which a gap space G2 extends radially inward between the lobe 58 and the hook 70, little or no radial gap exists between the radial outer-most contour of the lobe 118 and the hook 134 during mid-deployment. In some circumstances, the hooks 134 may be positioned at about the same distance from the central longitudinal axis for the medical device 110 as the radial outer-most contour of the lobe 118, and in other embodiments the hooks 134 may extend slightly farther in the radial direction compared to the radial outer-most contour of the lobe 118, or the radial outer-most contour of the lobe 118 may extend only a very small distance farther in the radial direction compared to the hooks 134 (e.g., a very small gap G2, if any, may exist). Without any gap similar to G2, as the lobe 118 begins to deploy from catheter 104, but before the proximal end 122 of the lobe 118 exits the catheter 104, the hooks 134 may readily contact, grab, or otherwise frictionally engage tissue within the treatment site, such as tissue of the LAA. At least in part because the hooks 134 of the lobe 118 are able to engage tissue prior to full deployment of the lobe 118 from the catheter 104, the lobe 118 is able to anchor to the target site during deployment so that during later stages of deployment, including full deployment of the medical device 110 from the catheter 104, the medical device is prevented from significant movement (including the “jumping” described above in connection with FIG. 3) during the remainder of deployment.

Thus, at least two benefits of the shape of the lobe 118 and position of the stabilizing wires 132 and hooks 134 relative to the lobe 118 include (i) the ability to treat a wider range of anatomies with a single size medical device 110 due to anchoring even during over-compression and (ii) the ability to deploy the medical device 110 with more stability due to early engagement of anchors with tissue at the target site. Although the figures show two distinct phases of deployment of the medical device 110 in which the hooks 134 are the radial outermost features of the medical device 110, it should be understood that this relationship may exist at all stages of deployment once the hooks 134 have exited the catheter 104.

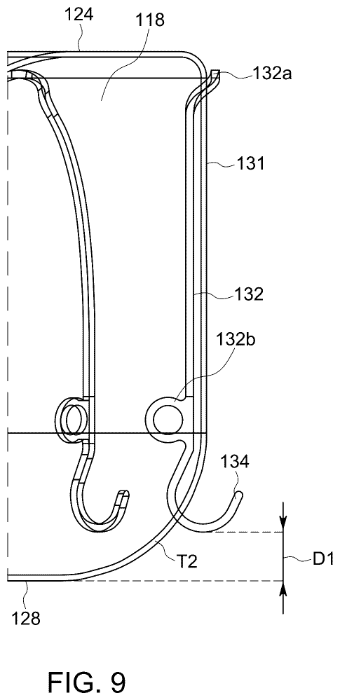

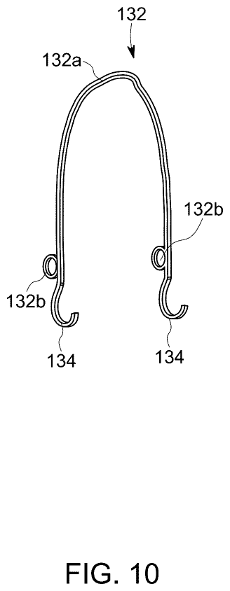

FIG. 9 shows an enlarged view of a portion of medical device 110 with a focus on the stabilizing wires 132 and hooks 134. In some examples, each stabilizing wire 132 may have a backing portion or a generally “U”-shaped proximal end 132a which, when coupled to the lobe 118, is positioned radially outward of the outer surface 131. The backing portion 132a may be positioned immediately adjacent to the proximal surface 124. The large majority of the length of each stabilizing wire 132 between the backing portion 132a and the hook 134 (which may also be referred to as an engagement portion) at the distal end may be positioned radially inside the outer surface 131. This may include a loop or eyelet 132b which may receive a suture or other fastener to fix the eyelet 132b to the outer surface 131 of the lobe 118, which as noted above may be a braided wire or mesh configuration. Options for structures of stabilizing wire 132 are described in greater detail in U.S. Patent Application Publication No. 2022/0280166, the disclosure of which is hereby incorporated by reference herein. FIG. 10 illustrates one example of a shape for stabilizing wire 132 isolated from other components of the medical device 110.

Referring again to FIG. 9, in some examples, the hook 134 may form a contoured section having a general “C” or hook shape starting at the distal end of the eyelet 132b and terminating at a position radially outward of the outer surface 131. In the illustrated example, the hook 134 may have a distalmost portion positioned between the eyelet 132b and the terminal end of the hook 134, such that the hook 134 extends proximally from the distalmost portion to the terminal end of the hook 134. This distalmost portion may be positioned radially outside of the outer surface 131 in the unbiased or set-shape of the medical device 110. In some examples, a distance D1 between the distalmost end of the hook 134 and the distal surface 128 of the lobe 118 may be up to about 2 mm, including up to about 1.5 mm, including between about 0 mm and about 1.0 mm. In some examples, this distance D1, for example which may be 1.0 mm, may be held constant even among different sizes of the medical device 110 (the different sizes corresponding to, for example, different diameters of the lobes 118 of the differently-sized devices in the unbiased condition or in the absence of applied forces). For example, if medical device 110 is offered in a 20 mm size (corresponding to a 20 mm diameter lobe 118), a 22 mm size (corresponding to a 22 mm diameter lobe 118), and a 25 mm size (corresponding to a 25 mm diameter lobe 118), each different size may still have a distance D1 of between about 0 mm and about 1.5 mm, including between about 0 mm and about 1.0 mm, including about 1.0 mm. This relationship may be at least partly responsible for the functionality described above in connection with FIGS. 7-8.

In some examples, the second transition T2 may have a contour length between the distal end of the substantially straight outer surface 131 and the beginning of the substantially straight distal surface 128, and the hook 134 may cross from inside the outer surface 131 to outside the outer surface 131 at a length that is about half (50%) or less than the contour length (measured starting from the substantially straight outer surface 131 toward the distal surface 128). In some examples, the point of crossing of the hook 134 may be between about 25% and about 40%, including about 30% or about 35%, of the contour length starting from the substantially straight outer surface 131 in the direction toward the distal surface 128. In some examples, the hook 134 may cross from inside the outer surface 131 to outside the outer surface 131 at any portion along the second transition T2.

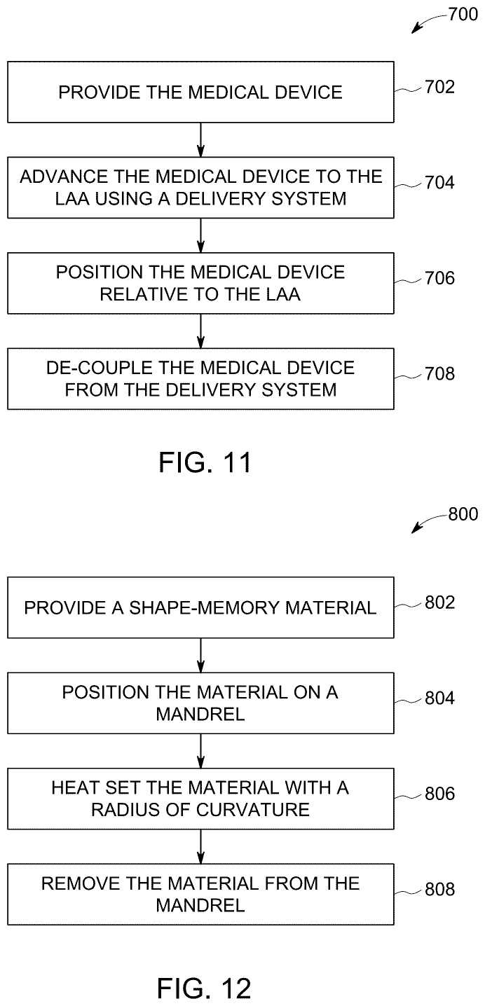

Turning now to FIG. 11, a flow diagram of an exemplary method 700 of using medical device 110 to occlude an LAA in a patient is depicted. In the exemplary embodiment, method 700 includes providing 702 a medical device. As described herein, the medical device includes a proximal and a distal end, wherein the proximal end comprises a disc and the distal end comprises a lobe, wherein the lobe comprises a proximal portion defining a proximal surface of the lobe, a distal portion defining a distal surface of the lobe, and a middle portion connecting and extending between the proximal portion and the distal portion, wherein a first transition between the proximal portion and the middle portion is curved and a second transition between the middle portion and the distal portion is curved, wherein the lobe has an expanded configuration when deployed at the target site and a reduced configuration for delivery to the target site, wherein the disc and lobe are connected by a connecting member, and a plurality of stabilizing wires coupled to the lobe at a radially outer surface of the middle portion, each stabilizing wire comprising a hook portion extending radially outward from the at least one lobe.

Method 700 also includes advancing 704 the medical device to the LAA using a delivery system including a catheter and a delivery cable, positioning 706 the medical device relative to the LAA to occlude blood flow to and from the LAA, and de-coupling 708 the medical device from the delivery cable to deploy the medical device.

Method 700 may include additional, alternative, and/or fewer steps, including those described herein. For example, in some embodiments, positioning 706 the medical device relative to the LAA includes placing the lobe of the medical device within the body of the LAA and the disc outside of the LAA abutted to the adjacent wall surrounding the opening of the LAA to prevent movement of the medical device towards the body of the LAA and to assist in sealing of the abnormality.

In some examples of method 700, as the medical device is deployed from catheter 104, once the hooks 134 have been released from the catheter 104, the hooks 134 are the most radially-outwardly positioned structure of the lobe 118 at all points until and including the full deployment of the lobe 118 into the LAA. Further, in some examples of method 700, a valley V is formed within the central portion 130 of the lobe 118 due to over-compression of the lobe 118 by tissue of the LAA, and despite the formation of the valley V, the hooks 134 are still in contact with tissue of the LAA for frictional engagement of the LAA.

Turning now to FIG. 12, a flow diagram of an exemplary method 800 of fabricating a medical device (e.g., medical device 110) is depicted. As described herein, the medical device includes a proximal and a distal end, wherein the proximal end comprises a disc and the distal end comprises a lobe, wherein the lobe comprises a proximal portion defining a proximal surface of the lobe, a distal portion defining a distal surface of the lobe, and a middle portion connecting and extending between the proximal portion and the distal portion, wherein a first transition between the proximal portion and the middle portion is curved and a second transition between the middle portion and the distal portion is curved, wherein the lobe has an expanded configuration when deployed at the target site and a reduced configuration for delivery to the target site, wherein the disc and lobe are connected by a connecting member, and a plurality of stabilizing wires coupled to the lobe at a radially outer surface of the middle portion, each stabilizing wire comprising a hook portion extending radially outward from the at least one lobe. In the exemplary embodiment, method 800 includes providing 802 a shape-memory material.

Method 800 may also include positioning 804 the shape memory material on a mandrel having a desired shape, size, and radius of curvature for the first transition and a radius of curvature for the second transition, heat setting 806 the shape-memory material to define the expanded preset configuration having the radius of curvature for the first transition and the radius of curvature for the second transition, removing 808 the shape-memory material from the mandrel to obtain the medical device in the expanded preset configuration.

Method 800 may include additional, alternative, and/or fewer steps, including those described herein. For example, in some embodiments, removing 808 the shape-memory material from the mandrel includes cooling the shape-memory material to room temperature.

In some examples, method 800 also includes coupling one or more stabilizing wires 132 to the lobe 118 of the medical device 110. For example, the “U”-shaped backing portion 132a may be positioned radially outward of the lobe 118 adjacent to the proximal surface 124, with two legs of stabilizing wire 132 extending distal to the “U”-shaped backing portion 132a passing through “cells” or openings of the braid so that the majority of the stabilizing wire 132 is positioned interior to the lobe 118. The backing portion 132a may be coupled to the braid, for example by one or more sutures. An eyelet 132b of the stabilizing wire 132, which may remain on the interior of the lobe 118, may also be sutured to the braid(s) of the lobe 118 to secure the eyelet 132b to the lobe 118. A portion of each hook 134 may be passed through a cell or opening of the braid so that at least a portion of the hook 134 protrudes to the exterior of the lobe 118. The positioning may be configured so that a distal-most end of the hook 134 is up to about 1.5 mm, including about 1.0 mm, from the distal surface 128 of the lobe 118.

While embodiments of the present disclosure have been described, it should be understood that various changes, adaptations and modifications may be made therein without departing from the spirit of the disclosure and the scope of the appended claims. Further, all directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure. It is intended that all matter contained in the above description or shown in the accompanying drawings shall be interpreted as illustrative only and not limiting. Changes in detail or structure may be made without departing from the spirit of the disclosure as defined in the appended claims.

Many modifications and other embodiments of the disclosure set forth herein will come to mind to one skilled in the art to which this disclosure pertains having the benefit of the teachings presented in the foregoing descriptions and the associated drawings. Therefore, it is to be understood that the disclosure is not to be limited to the specific embodiments described and that modifications and other embodiments are intended to be included within the scope of the appended claims. Although specific terms are employed herein, they are used in a generic and descriptive sense only and not for purposes of limitation.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. An occluder for occluding a left atrial appendage (“LAA”), the occluder comprising:
a lobe sized and shaped for positioning within the LAA, the lobe having a proximal end defining a proximal surface of the lobe, a distal end defining a distal surface of the lobe, and a middle portion connecting and extending between the proximal end and the distal end, wherein the lobe has an expanded condition in the absence of applied forces and a collapsed condition for delivery to the LAA; and
a plurality of stabilizing wires coupled to the lobe, each of the plurality of stabilizing wires having a hook at a distal end thereof, each hook having a terminal end positioned radially outwardly of the lobe in the expanded condition of the lobe and being configured to frictionally engage tissue of the LAA,
wherein in the expanded condition of the lobe, each hook has a distalmost point, the distalmost point of each hook being positioned a spaced axial distance from the distal surface of the lobe in the expanded condition of the lobe, the spaced axial distance being between about 0 mm and about 1.5 mm.

2. The occluder of claim 1, wherein the spaced axial distance is about 1.0 mm.

3. The occluder of claim 1, wherein a transition between the middle portion and the distal end of the lobe is curved, the curve of the transition having a contour length measured between (i) a point at which a distal end of the middle portion joins a proximal end of the curved transition, and (ii) a point at which a distal end of the curved transition joins the distal surface of the lobe.

4. The occluder of claim 3, wherein in the expanded condition of the lobe, the hook crosses from an interior of the lobe to an exterior of the lobe at a crossing point, the crossing point being positioned between about 25% and about 50% of the contour length.

5. The occluder of claim 4, wherein the transition has a radius of curvature between about 0.075 inches (1.905 mm) and about 0.125 inches (3.175 mm).

6. The occluder of claim 1, wherein the occluder includes a disc at a proximal end of the occluder, and a connecting member connects the disc and the lobe.

7. The occluder of claim 1, wherein in a compressed state of the lobe, a valley is formed within the middle portion of the lobe, and in the compressed state, the hook extends farther radially outward from a central longitudinal axis of the lobe than does any structure of the lobe.

8. An occluder for occluding a left atrial appendage (“LAA”), the occluder comprising:
a lobe sized and shaped for positioning within the LAA, the lobe having a proximal end defining a proximal surface of the lobe, a distal end defining a distal surface of the lobe, and a middle portion connecting and extending between the proximal end and the distal end, wherein the lobe has an expanded condition in the absence of applied forces and a collapsed condition for delivery to the LAA; and
a plurality of stabilizing wires coupled to the lobe, each of the plurality of stabilizing wires having a hook at a distal end thereof, each hook having a terminal end positioned radially outwardly of the lobe in the expanded condition of the lobe and being configured to frictionally engage tissue of the LAA,
wherein a transition between the middle portion and the distal end of the lobe is curved, the curve of the transition having a contour length measured between (i) a point at which a distal end of the middle portion joins a proximal end of the curved transition, and (ii) a point at which a distal end of the curved transition joins the distal surface of the lobe,
wherein in the expanded condition of the lobe, the hook crosses from an interior of the lobe to an exterior of the lobe at a crossing point, the crossing point being positioned between about 25% and about 50% of the contour length.

9. The occluder of claim 8, wherein in the expanded condition of the lobe, the crossing point is positioned between about 25% and about 40% of the contour length.

10. The occluder of claim 9, wherein in the expanded condition of the lobe, the crossing point is positioned between about 30% and about 35% of the contour length.

11. The occluder of claim 8, wherein in the expanded condition of the lobe, each hook has a distalmost point, the distalmost point of each hook being positioned a spaced axial distance from the distal surface of the lobe in the expanded condition of the lobe, the spaced axial distance being about 1 mm.

12. The occluder of claim 8, wherein the transition has a radius of curvature between about 0.075 inches (1.905 mm) and about 0.125 inches (3.175 mm).

13. The occluder of claim 8, wherein the occluder includes a disc at a proximal end of the occluder, and a connecting member connects the disc and the lobe.

14. The occluder of claim 8, wherein in a compressed state of the lobe, a valley is formed within the middle portion of the lobe, and in the compressed state, the hook extends farther radially outward from a central longitudinal axis of the lobe than does any structure of the lobe.

15. A method for occluding a left atrial appendage (“LAA”), the method comprising:
positioning an occluder in a collapsed condition within a delivery catheter, the occluder including:
a lobe having a proximal end defining a proximal surface of the lobe, a distal end defining a distal surface of the lobe, and a middle portion connecting and extending between the proximal end and the distal end; and
a plurality of stabilizing wires coupled to the lobe, each of the plurality of stabilizing wires having a hook at a distal end thereof;
advancing the occluder into or adjacent to the LAA while the occluder is collapsed within the delivery catheter;
transitioning the lobe into a partially-expanded state in which the distal end of the lobe has exited the delivery catheter and has begun to self-expand while the proximal end of the lobe remains collapsed within the delivery catheter;
while the lobe is in the partially expanded state, contacting the hook with tissue of the LAA; and
after contacting the hook with tissue of the LAA, fully deploying the occluder from the delivery catheter.

16. The method of claim 15, wherein between the partially expanded state of the lobe and the full deployment of the occluder from the delivery catheter, the hook remains in contact with the tissue of the LAA.

17. The method of claim 15, wherein after fully deploying the occluder from the delivery catheter, compression on the lobe from tissue of the LAA creates a valley within the middle portion of the lobe.

18. The method of claim 17, wherein while the valley is created within the middle portion of the lobe, the hook remains in contact with the tissue of the LAA.

19. The method of claim 15, wherein fully deploying the occluder from the delivery catheter includes deploying a disc of the occluder from the delivery catheter, the disc of the occluder being coupled to the lobe of the occluder by a connecting member.

20. The method of claim 19, wherein upon deploying the disc of the occluder, the disc at least partially covers an ostium of the LAA.
